(19) United States
(12) Patent Application Publication
(10) Pub. No.: US 2008/0031938 A1
(43) Pub. Date: Feb. 7, 2008

(54) BIOCIDAL MATERIALS FOR TREATMENT AGAINST PATHOGENS

(76) Inventors: Clay E. Easterly, Konxville, TN (US); Guy D. Griffin, Oak Ridge, TN (US); Michael Z. Hu, Knoxville, TN (US); Arpad A. Vass, Oak Ridge, TN (US)

Correspondence Address: AKERMAN SENTERFITT, 222 LAKEVIEW AVENUE, 4TH FLOOR, WEST PALM BEACH, FL, 33401, US

(21) Appl. No.: 11/748,649

(22) Filed: May 15, 2007

Related U.S. Application Data
(63) Continuation-in-part of application No. 10/917,047, filed on Aug. 11, 2004.

(51) Int. Cl. A61K 9/127; A61K 33/38

(52) U.S. Cl. 424/450; 424/618; 424/630; 424/644; 977/907

(57) ABSTRACT

Applicant's present invention is a biocidal material for in vivo use for treatment of pathogenic infections comprising nanoparticles or nanostructures of biocidal material encapsulated within a liposomal carrier

### CROSS-REFERENCE TO RELATED APPLICATIONS

This application is a continuation-in-part of U.S. patent application Ser. No. 10/917,047, filed on Aug. 11, 2004, entitled “Biocidal Materials for Treatment Against Pathogens” which is incorporated herein by reference in its entirety.

The United States government has rights in this invention pursuant to contract no. DE-AC-05-00OR22725 between the United States Department of Energy and UT-Battelle, LLC.

### FIELD OF THE INVENTION

The present invention relates to the treatment of living systems infected by pathogens, and more particularly relating to biocidal agent materials and their in vivo use for treatment of living systems infected by pathogens.

### BACKGROUND OF THE INVENTION

Historically, metals proved effective in the treatment of human infections like venereal diseases, fungal and protozoal diseases and dysentery. Early pharmacologists coined the term “oligodynamic action” to refer to the relative efficacy of metal compounds as antibacterial agents at very low concentrations. Mercury as chloride and silver as nitrate were identified as the most efficacious of the early metal pharmaceuticals. They inhibited growth of a wide range of gram-positive and gram-negative microorganisms at concentrations of less than 1 ppm (Lansdown 2002). Most metal-based antibacterial compounds were superseded by sulphonamides and penicillins upon their introductions. Following the development of modem antibacterial chemotherapy, (i.e. penicillins) metal-based anti-infective preparations received little additional attention. In more recent times, many hospitals have adopted the use of electrolytically deposited copper and silver in the parts per billion range as disinfectants in hot potable water.

Silver is gaining acceptance in antimicrobial preparations in the management of burns. The recent reporting that silver ions, at low concentrations, induce a massive proton leakage through bacterial cell membranes and that the antimicrobial activity derives primarily from silver oxides provides credence to the long held observations of silver ions effectiveness at low concentrations. Interest in silver-based antimicrobials for topical application has persisted due to their proven efficacy against wound infections, their relatively low toxicity and the introduction of silver sulphadiazine, which controls the delivery of silver ions to skin wounds, most notably burns.

A growing interest has focused on the microbial effect of nanocrystalline silver, as used in burn dressings. The interest lies in the fact that efficacy is produced at substantially lower silver concentrations than with the standard, silver sulphadiazine or electrodeposited silver. Djokic and Burrell (1998) studied the antimicrobial characteristics of silver nanocrystal films of various origin. Their results suggest that the essential factor leading to antimicrobial effect is the presence of oxide(s) in the silver material. Fan and Bard (2002) investigated the antimicrobial silver films made by sputtering silver in the presence of low concentrations of oxygen using chemical, electrochemical, gravimetric and microscopic studies. Their results suggest that the antimicrobial films contain Ag(0) and Ag(I) in different proportions, the form of Ag(I) being Ag₂O, and/or AgOH. Djokic et al (2001), using electrochemical analysis of bioactive films produced by sputtering in the presence of argon and oxygen, conclude that the films contain oxidized silver species Ag₂O and Ag₂CO₃ and metallic silver. In the recent past, the above-cited investigations have provided mechanistic credence to the long-held empirical observation that silver is an effective biocide.

### OBJECTS OF THE INVENTION

Accordingly, it is an object of the present invention to provide a biocidal agent for the in vivo treatment of living systems infected by a pathogen.

It is another object of the present invention to provide a biocidal agent for the in vivo treatment of living systems infected by biological warfare agents.

These and other objects, features and advantages of the present invention will become apparent after a review of the following detailed description of the disclosed embodiments and the appended claims.

### SUMMARY OF THE INVENTION

In accordance with one aspect of the present invention, the foregoing and other objects are achieved by a biocidal agent for in vivo use for treatment of pathogenic infections comprising nanoparticles or nanostructures of biocidal material encapsulated within a liposomal carrier.

### BRIEF DESCRIPTION OF THE DRAWINGS

In the drawings:

FIGS. 1a and 1b show electron micrographs of “naked” silver nanocrystals <10 nm, formed by a membrane-assisted thermal electro-chemical method.

FIG. 2 shows a spectrometric analysis of silver concentration in sol prior to incorporation into liposomes using silver nitrate standard.

FIGS. 3a and 3b show electron micrographs of 85 nm long-lived liposomes.

FIG. 4 is a photograph (100X) of an amoebae culture infected with Legionella and subsequently treated with liposomes.

FIG. 5 is a photograph (100X) of an amoebae culture infected with Legionella, but not treated with liposomes.

FIG. 6 is a photograph (400X) of macrophages of J774 line infected with Legionella and treated with liposomes.

FIG. 7 is a photograph (400X) of macrophage of J774 line infected with Legionella but not treated with liposomes.

For a better understanding of the present invention, together with other and further objects, advantages and capabilities thereof, reference is made to the following disclosure and appended claims in connection with the above-described drawings.

### DETAILED DESCRIPTION OF THE INVENTION

Applicant's invention offers a new therapy that could be used as an in vivo medical countermeasure after a terrorist release of biological warfare agents designed or selected to be antibiotic resistant. Alternatively, applicant's invention can be used as an in vivo treatment against other types of pathogenic infections in living systems, both mammalian and non-mammalian systems that are resistant to conventional antibiotic therapy either by natural adaption, human selection or human engineering. Applicant's invention makes use of biocidal materials normally thought of as useful outside the body by conveying them into the body to the site of inflammation caused by pathogens. Pathogen is defined as any disease-producing organism including bacteria, antibiotic-resistant bacteria, viruses, fungi, nosocomial infectious agents, biological warfare agents that are resistant to antibiotic therapy either by natural adaption, human selection or human engineering, etc. Applicant's present invention offers a biocidal agent or a “kill system” utilizing long-lived liposomes functioning as liposomal carriers loaded with a biocidal material such as silver nanoparticles injected into the blood of a living system to deliver the biocidal material to the inflammation site caused by a pathogenic infection, (infection from a pathogen), within the living system. Applicant's invention is most effective against pathogens that replicate within macrophages as the action of applicant's biocidal material takes place intracellularly, within the macrophage. Because the therapeutic biocidal material may remain in the blood stream for up to a few days, the therapy may have prophylactic capacity and be used in battlefield conditions. Long-lived liposomes as liposomal carriers of a particular formulation, such as polyethylene glycol (PEG)-coated liposomes approximately 100 nm in size (or less than 150 nm in size), accumulate preferentially at inflammation sites along with neutraphils from the blood system. The macrophages present at inflammation sites will engulf both invading pathogens and the liposomes. Replication of the pathogens occurs within the macrophages. Degradation processes within the macrophages dissolve the liposomes, freeing the biocidal material proximate to the engulfed pathogens for destruction of the pathogens. The rate of degradation increases as the pH within the macrophage is decreased. Applicant's invention is most effective against pathogens that replicate within macrophages as the action of applicant's biocidal material takes place intracellularly, within the macrophage. For therapeutic purposes, nanoparticle silver is the preferred biocidal material for Applicant's invention, particularly nanoparticle colloidal silver. By using silver nanoparticles rather than silver chloride, silver nitrate, or any of the many silver compounds known to be toxic to bacteria, Applicant's present invention provides an improvement in therapy. This improvement is because the silver nanoparticle dissolves in the cellular medium of the infected cell. This dissolution is not instantaneous or very fast like silver chloride or silver nitrate because of the solubility limit of the silver nanoparticle in the cellular medium. If one of the silver salts were to be used, then once the liposome is destroyed, the entire quantity of silver salt would be available for reactions within the host cell, killing proximate bacteria and damaging the host cell and its nearby neighbor. In contrast, silver nanoparticles dissolve more slowly as they provide a constant supply of silver ions. Thus, as a macrophage continues to ingest additional bacteria or whatever pathogens are present, these pathogens can be exposed to more silver as it dissolves from the nanoparticles. It is important at this point that the concentration of the biocidal material be tailored so as to not kill the host cell along with the bacteria. By using the nanoparticle, the concentration of biocidal material being released would be therapeutic rather than harmful to the host cell. In Applicant's invention, it is vitally important that the nanoparticles remain the size of their original formation and not increase in size during the period between formation and actual use as a therapeutic material. The nanoparticles must be of proper size to release ions resulting in an adequate concentration to kill engulfed bacteria or pathogens but not kill the host cells. The liposomes must effectively incorporate the nanoparticles and simultaneously lend themselves to inclusion into phagocytic cells, either by membrane fusion or by engulfment by phagocytic mechanisms. The liposomes must release their biocidal kill package intracellularly.

It is the engineering of pure metallic silver to “naked” nanoparticles that can deliver a steady stream of silver ions to biological targets that makes Applicant's invention innovative, safe and successful. With the nanoparticles, safety and efficacy are intertwined. In normal biological media, the nanoparticles have a relatively low solubility limit. But, the environment in the phagosome of a macrophage (that has engulfed pathogens as well as silver nanoparticle-containing liposomes) rapidly goes from a pH of about 6.8 to 4.5, thus drastically enhancing the solubility limit of the silver nanoparticle. Nanoparticle engineering, coupled with the immune system's characteristics, thus transforms a relatively inert material into a proximity bomb. Applicant's kill system employs a great variety of killing biocides in addition to silver nanoparticles in order to provide a defense that employs a wide variety of killing mechanisms. For example, additional kill systems employ biologically active elements such as copper, nickel, zinc, other elements in the periodic table as well as compounds such as silver iodide, mercury compounds, compounds using ozone, biologically active structures such as proteins or enzymes, and nanosized chemical structures such as carboxyfullerenes and the non-ionic surfactant nanoemulsion designated 8N8, etc. Biologically active elements or nanostructures are elements from the periodic table or nanosized biological structures such as proteins and enzymes that function to enhance or inhibit a biological mechanism or process.

Liposomes have been widely investigated as targeted drug carriers in infectious diseases. They have been shown to localize selectively at infected target sites where inflammation is present in a variety of experimental models of infection for a variety of organs. The localization at inflamed infection sites is dependent on the inflammatory response. Several studies have shown an improved target site localization of liposomes coated with poly(ethylene)glycol (PEG), also known as sterically-stabilized liposomes (SSLs), compared to conventional liposomes that lack the PEG coating. More generally, as known in the art, SSLs are polymer coated liposomes, wherein the polymer is covalently bound to one of the phospholipids and provides a hydrophilic cloud outside the vesicle bilayer. Besides PEG, other hydrophilic polymers can be used with the present invention, including, but not limited to, polyvinylalcohol, polyvinylpyrolidone, polyacrylamide, starches, and other polysaccharides, as well as methoxypolyethylene glycol (mPEG) poly(acryloylmorpholine), poly(vinylpyrrolidone), and poly(2-oxazoline). It is generally accepted that the higher degree of localization is enabled by the prolonged circulation time of SSLs, also known as “stealth” liposomes. The increased average liposome concentration in the capillaries coupled with increased capillary permeability at the inflamed area yields increased exposure of the target site to the liposomes. Moreover, permeability studies in tumor tissue suggest that the PEG coating itself can promote target localization. Schiffelers et al. (1999 Biochimica et Biophysica Acta; 2001 Pharm. Research; and 2001 JAC Reviews, all incorporated herein by reference) have developed relationships between liposomal circulation time and target site localization and they investigated the effect of the PEG coating itself by comparing the circulation kinetics and target localization of long-circulating “PEG-free” liposomes and SSLs in experimental rat Klebsiella pneumoniae pneumonia infections. Schiffelers et al. found a positive correlation between circulation times and target localization from experiments performed on the effect of liposome size on circulation kinetics and biodistribution of SSL. SSL having particle mean sizes of 280 or 360 nm showed an approximately much lower target localization compared to PEG liposomes of 100 nm size. This may be attributed to differences in circulation times as 100 nm sized liposomes are cleared more slowly from the blood stream compared to the liposomes with larger mean sizes.

For several decades, silver in combination with copper, has been used to purify drinking water. A substantial number of hospitals have begun to take advantage of this recognized low-concentration effect on microorganisms and have adopted copper and silver ions, electrolytically produced to maintain Legionella-free potable hot water systems. Lin et al. (1996) have determined that copper and silver combinations at part per billion levels (40 ppb silver and 400 ppb copper) contribute to synergistic action on Legionella when each is above a certain concentration. Cell penetration by silver is considered the principal objective in the development of copper/silver ionization systems. Positively charged copper ions form electrostatic bonds at negatively charged sites on bacterial cell walls, and the resulting damage permits the greater uptake of silver ions. A recent report has investigated the cell wall mechanism using Vibrio cholerae, and found that low concentrations of Ag+ induce a massive proton leakage through the membrane, which results in complete deenergization, and, with a high degree of probability, cell death.

Applicant's present invention brings forth this new therapy that is useful as an in vivo medical countermeasure after a terrorist release of biological warfare agents designed to be antibiotic resistant. Alternatively, the present invention is also useful on nosocomial infectious agents that have become resistant to current antibiotics. Because the sterically-stabilized liposomes (SSLs) naturally accumulate at the sites of inflammation, the therapy is broad-based and does not require identification of the pathogen.

Inhalation exposure is by far the most likely scenario with the greatest number of potential victims in a terrorist attack. Once engulfed, macrophage-mediated pathogens (most biological warfare agents are of this type) overcome the destructive nature of the macrophage and begin to replicate within the phagosome. This process continues until the pathogens rupture the host macrophage and are released, either becoming engulfed by other macrophages where the process repeats or by causing massive lung inflammation thereby gaining access to the blood stream. Once in the blood stream, pathogens disseminate throughout the body eventually causing irreversible damage and then death of the host. For purposes of Applicant's proof-of-principle, Legionella was chosen as the model biological warfare system. This microbe, Legionella pneumophila, the causative agent of Legionnaires' disease and related respiratory ailments is a facultative intracellular pathogen similar to many biological warfare agents. The bacteria are able to infect, multiply within, and kill human macrophages in a fashion similar to biological warfare agents. In the examples, included herein below, two phagocytic organisms were used to demonstrate the therapeutic efficacy of Applicant's present invention: J774, a mouse macrophage cell line, and amoebae, which are noted for their phagocotic activity.

Applicant's present invention comprises a biocidal material in the form of nanoparticles, such as colloidal silver, silver iodide, copper, mercury compounds, compounds using ozone, etc., encapsulated inside a liposomal vesicle. Since the sizes of liposomes are in the range of 80-90 nm (generally less than 150 nm), the nanoparticles must be much smaller than this dimension in order to be reasonably trapped inside the liposome vesicles. In Applicant's experiments, silver nanoparticles were used (<10 nm) for encapsulation in molecular self-assembly such as liposomes to create a package that is suitable for targeted therapy due to the antibacterial property of nanocrystal silver. An aqueous silver nanoparticle sol is preferred for compatibility with the liposome formation conditions. To assure the maximum biological reactivity in terms of nanoparticle solubility to provide a constant concentration of silver ions after the liposomes are engulfed by macrophages and encapsulated within a phagasome, a nanoparticle in the size range of 1-10 nm was developed and is preferred. Typically, the production of monodispersed silver nanoparticles involves the use of steric capping molecules or stabilizers (such as trialkylphosphine/amine, alkanethiols, long-chain unsaturated carboxylates, CS₂, quaternary ammonium disulfide) to control the size and stabilize the nanoparticles. But to avoid any possible effect of surface modifying molecules on the effectiveness of silver nanoparticles in killing bacteria, mostly due to solubility of silver in aqueous solution, the silver nanoparticles were developed using membrane-assisted thermal electrochemical method. This membrane-assisted thermal electrochemical method produces the correct size of nanoparticles that are “naked”; that is, the nanoparticles are clean containing pure metallic silver nanocrystals with no organic molecules existing on the surfaces of the nanocrystals or nanoparticles or in the bulk aqueous solution background. Applicant's nanocrystals are also free of oxidized species. These silver nanoparticles require no stabilization. Commercially available nanoparticles of sufficient size so to be encapsulated within the liposomal vesicle have the potential for use with Applicant's invention if they have similar solubility properties and biological activity to those preferred by Applicant, discussed below.

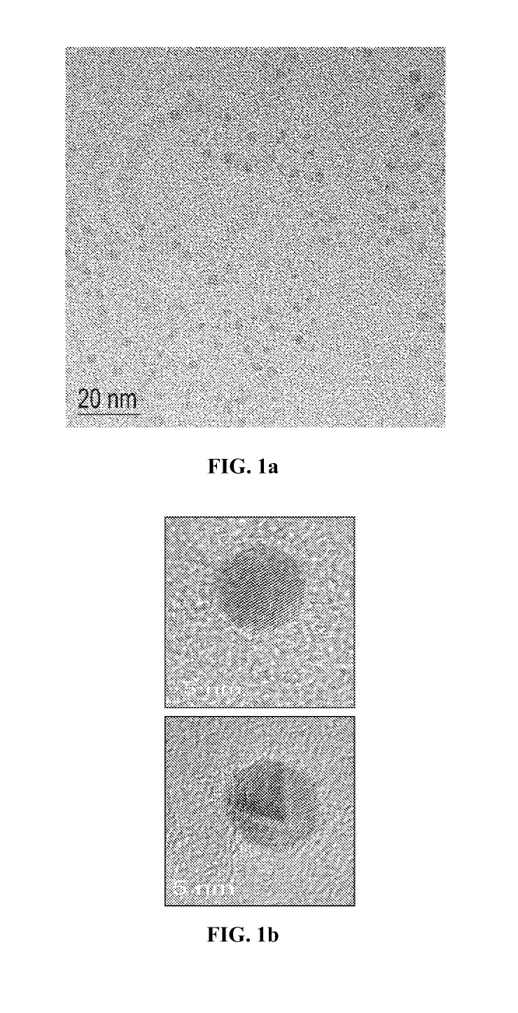

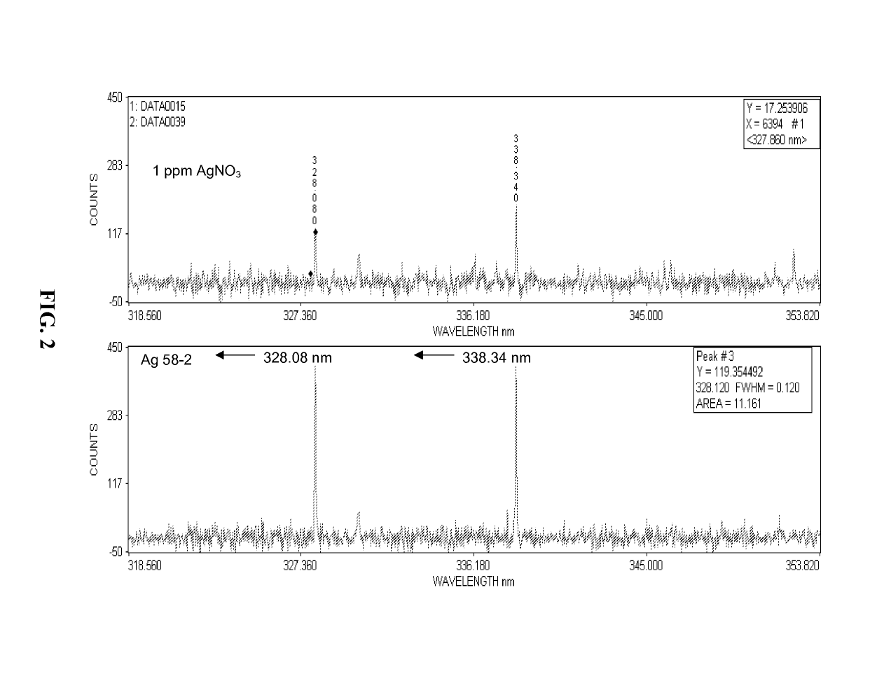

Nanoparticle preparation. Nanoparticles of pure silver nanocrystals were produced that were 5-10 nm in size with a polydispersity index of about 0.3 (see FIG. 1). Average size was determined using dynamic light scattering and Transmission Electron Microscopy (TEM). These pure silver nanoparticles were highly effective in tests with Legionella alone and with cells infected with Legionella. This finding is in contrast with the discussion of the reports by Fan and Bard (2002), and by Djokic et al. (2001) who suggest that the silver nanoparticle should be an oxidized species to be biologically active. Djokic et al. (2001) use indirect means to estimate the size of their silver oxide nanoparticles to be in the range of 4-40 nm. However, their electron micrographs of their oxidized silver films suggest particles at least 100 times larger. The nanoparticles of the present invention were small enough to dissolve at a rate adequate to provide a killing concentration of silver ions. The effective concentration of silver in the sol in Applicant's experiments was approximately 4.5 ppm (see lower FIG. 2). FIG. 2 shows a comparison of Applicant's effective concentration of silver in sol prior to incorporation into liposomes (lower FIG. 2) as compared to the silver standard solution shown in upper FIG. 2 that is 1 ppm, used for calibration of the spectrometer. For in vivo use, the effective concentration of silver in the sol or effective concentration of the biocidal material within the liposome is of a sufficient concentration to be effective within the living system, within the intracellular medium without being toxic to the living system host.

The nanoparticles used in Applicant's invention were prepared by a membrane-assisted thermal electrochemical method that produces colloidally stable aqueous sols/suspensions of naked nanoparticles of less than 10 nm, without the need of surfactant or polymer dispersants, such as PVP or PVA, used in the existing literature. The thermal electro-chemical process began with a reaction vessel containing reaction solutions (typically distilled, deionized and filtered (0.02 micron) pure water) heated to a target temperature (30-100° C.), then two silver electrodes enclosed by dialysis membrane tubes were placed in deionized water with a small distance apart (alternatively, cathode could be other metals such as Pt). The silver anode served as the metal (ion) source for the silver nanoparticle formation in water solution. A low direct current (DC) voltage (0-50) was applied on the electrodes. When electric current passed through the silver anode, some silver atoms at the interface with water lost an electron and became ions. In this electrochemical process, some of the silver ions in close proximity to the anode accept electrons from the current passing through and reduce to metallic atoms, which attract each other by van der Waal's forces of attraction and thus form small metallic nanoclusters. Overall, the electric current flow causes Ag⁰ (metal) and Ag⁺(ions) to migrate from electrode into the deionized water. The reaction vessel was brought up to the target temperature while being immersed in a water or oil thermostat bath. Direct electrical current, 10-35 volts was applied to the silver electrodes for 20 minutes or longer, until deep golden yellow color was achieved. It takes longer with larger vessels and more separation of electrodes. Initially the current was 11 milliamps (ma) which reduced to 6 ma midway through the procedure as the power supply attempted to maintain constant voltage.

Visible crystals/particles formed quickly on electrodes and crystals aggregated into chains and fractal tree structure between the two electrodes. Microarcing could occur, resulting in a lot of large particles. Stirring of the reaction solutions reduces formation of visible crystals significantly. Without the dialysis membrane tube enclosed around the electrodes, bright golden yellow solutions can be produced under appropriate conditions. However, large aggregate or crystal particles (originally formed on/near the electrode surfaces) tend to be mixed into the yellow solution containing small (<10 nm) nanocrystals. Larger nanoparticles, 100 nm, form when the dialysis membrane is in a “U” configuration where it is not tied to separate the anode from cathode. When tied, nanoparticles of ˜1 to 10 nm are formed. The reaction rate is temperature dependent. No reaction was observed at 3° C., and the nanoparticle formation rate increased progressively up to 95-98° C. The reaction rate also increased with decreasing electrode separation and with increasing voltage.

Silver nanoparticle sols were stored in low potassium scintillation vials because storage in other glass containers resulted in degradation of the size. This phenomenon is thought to be associated with decay of K-40 in other containers, leading to radiation-induced deterioration of the 5-10 nm self-assemblies to particles larger than 100 nm. In Applicant's experiments, 5-10 nm samples were monitored using visible-uv absorption. The prepared sols have remained consistent in size and concentration for at least two months.

Liposome preparation. Methods for preparation of liposomes described by Schiffelers et al., (2001), incorporated herein by reference, were followed. Preferential localization of liposomes at sites of inflammation has been demonstrated in a variety of experimental models. For intravenously introduced liposomes, it is generally accepted that the prolonged circulation time of poly(ethylene) glycol (PEG)-coated liposomes as compared to conventional non-coated liposomes is a result of the PEG coating. Schifflelers et.al. (2001) incorporated two antibiotics into their liposomes. The approach of the present invention was to prepare 5-10 nm silver nanocrystals and incorporate them into the liposomes.

Liposomes were prepared using procedures described in the literature. Briefly, the following lipids were dissolved in a mixture of chloroform/methanol at the indicated molar ratios: partially hydrogenated egg phosphatidyl choline, cholesterol, and 1,2-distearoyl-sn-glycero-3-phosphoethanolamine-N-(polyethylene glycol 2000) at a molar ratio of 1.85:1:0.015. The solvents were evaporated under nitrogen and the lipids dried and redissolved in 2-methyl-2-propanol, followed by shell-freezing and subsequent lyophilization. The resulting film of lipid on the glass lyophilization bottle was hydrated in the silver nanoparticle sol at 40-45° C. for 1 hour. Following hydration, the lipid dispersion was sonicated, at 40-45° C. for 15 minutes, (Sonicator Model 112 SPIG, 80 KC, 80 Watts, Laboratory Supplies Co., Hicksville, N.Y.).

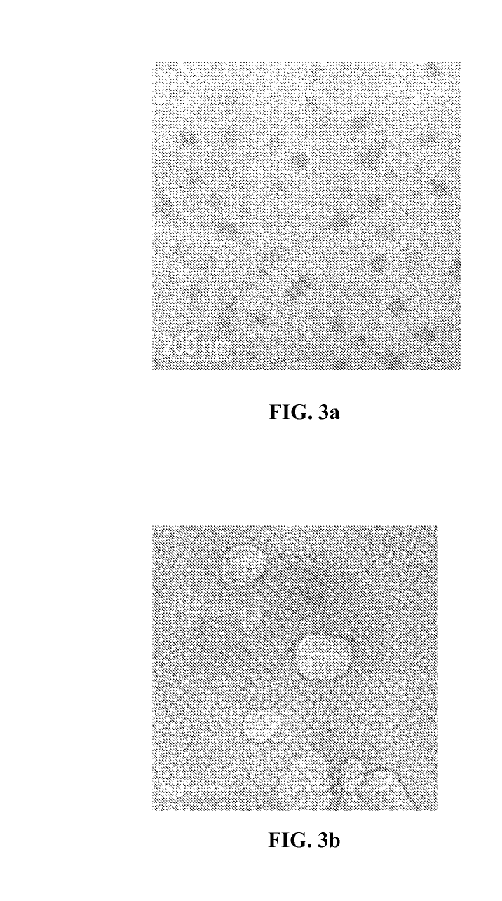

Using dynamic light scattering, the average size of the liposomes was 85 nm with a polydispersity of 0.3. FIG. 3 contains three images taken with TEM.

Liposomes were then separated from unincorporated silver sol with a Sephadex G-25 column using distilled deionized water as the elutant. After passage of the liposomes, the next 10 ml were collected. Analysis of this volume by visible-uv absorption indicated that the silver concentration was 40× less than the concentration in the silver sol used to hydrate and fill the liposomes. Thus the non-encapsulated silver in the liposome preparation is less than approximately 0.1 ppm.

Biotesting of nanocrystalline silver and nanocrystalline silver-containing liposomes. The first step was to evaluate the concentration of silver solution required to kill cultures of Legionella. Cultures of Legionella were grown to a concentration of 10⁶/ml and exposed to concentrations of silver sol using the neat preparation. With 1:1 mixture of Legionella and nanocrystalline silver (about 4.5 ppm), after 30 minutes, all Legionella were killed as determined by reexamination of plates incubated for 48 hours after exposure to determine if any breakthrough growth occurred; none did. The use of a 1:10 dilution of the silver sol produced about 99% kill after 1 hour, but even after 48 hours, the kill was not 100%. Replication yielded substantially the same results. Thus, nanocrystalline silver in the low ppm range provided good killing of Legionella.

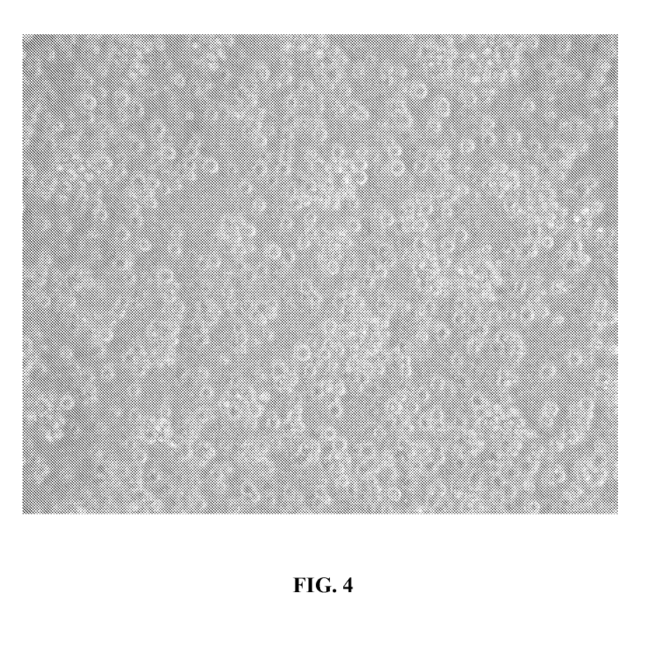

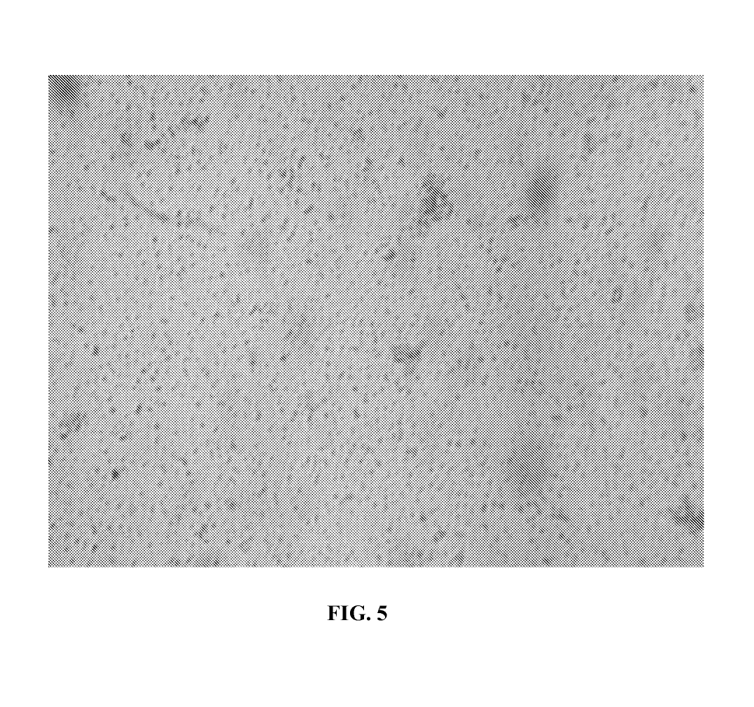

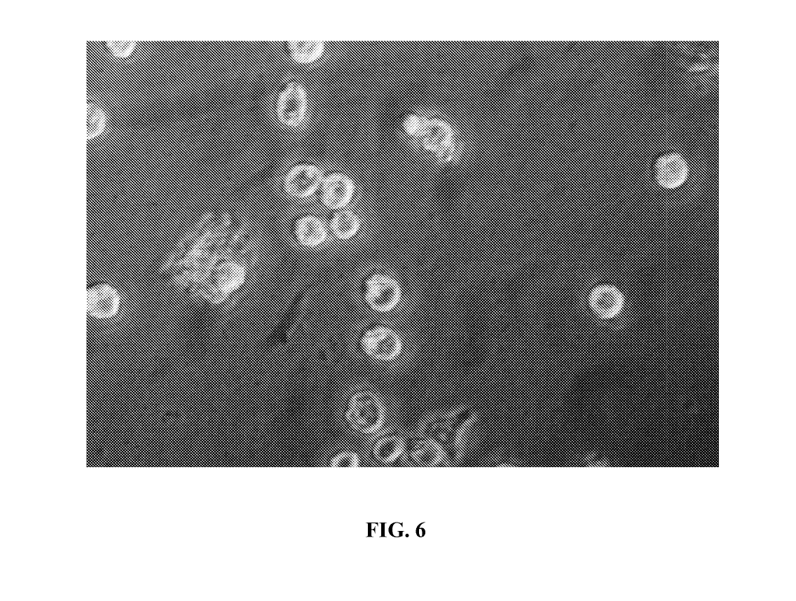

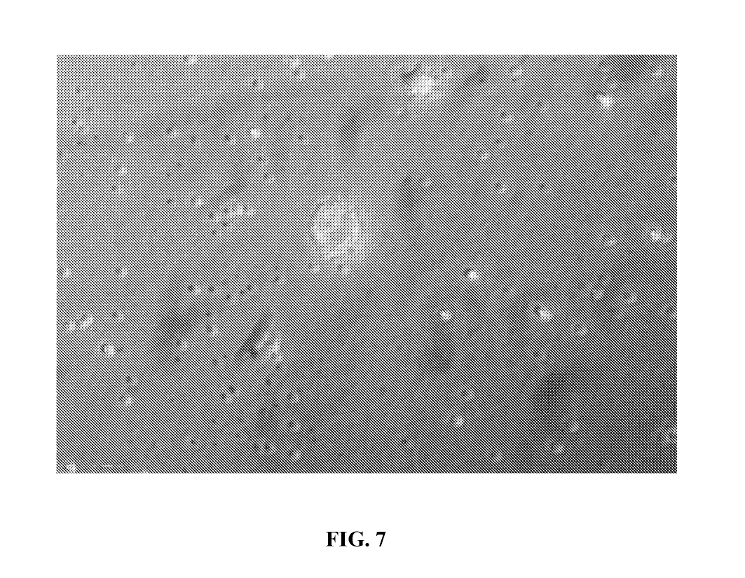

The next step was the proof-of-principle experiment. This involved the infection of cells with Legionella and subsequent exposure of the cells to the liposomes containing the silver nanocrystals. Prior experiments established the concentration of silver which was effective against Legionella and did not exhibit toxic effects on the cell lines. The proof-or-principle experiment was developed around the liposomes, described earlier; as produced, they had an average 85 nm particle size and contained an effective concentration of approximately 4.5 ppm of silver. Experiments were set up in duplicate with two flasks of amoebae Acanthamoebae royreba and line J774 mouse macrophages set up as controls (nothing added). Both cultures were plated initially at densities of 10⁴ cells/ml. To the eight test culture flasks (four of Acanthamoebae and four of J774), 10 μl of a 1.2×10⁸/ml culture of viable Legionella (final concentration 1.2×10⁶/flask) was added 2 days prior to treatment with the liposomes in order to ensure adequate time for phagocytosis. (Phagocytosis is the process by which the macrophage or amoebae engulf the bacteria.) After two days, the media was changed in all flasks and 160 μl of the liposome preparation was added to two flasks of each set, resulting in 2 control, two Legionella-infected and two Legionella-infected plus liposome treated flasks for each cell line. These flasks were monitored daily over a period of two weeks and the media was changed as needed. After media changes, liposomes (160 μl) were re-added to ensure adequate treatment. After 8 days, the treated amoebic cultures were fully recovered while the untreated had no obvious survivors. By day 12, the treated J774 line was essentially filly recovered while the untreated had no obvious survivors. Both J774 and amoebae cultures that were infected with Legionella and subsequently treated with the liposomes recovered. Those cultures that did not receive liposomes were completely destroyed (see FIG. 4, FIG. 5, FIG. 6 and FIG. 7).

Applicant's invention demonstrates that silver nanoparticles encapsulated into liposomes act as a “stealth” bacteriocidal agent destroying bacteria in growing mammalian cells, and in a non-mammalian phagocyosing line, the amoebae.

Another application of Applicant's invention comprises encapsulating nano-sized biologically active structures such as enzymes into long-lived liposomes. For example, an antrax-killing phage lytic enzyme has been found to specifically kill anthrax, but not anything else. Encapsulating this enzyme within long-lived liposomes as a biocidal agent offers a unique treatment that could deliver phage lytic enzyme to sites of infection-caused inflammation against anthrax that would not cause side effects.

While there has been shown and described what are at present considered the preferred embodiments of the invention, it will be obvious to those skilled in the art that various changes and modifications can be made therein without departing from the scope of the invention defined by the appended claims.

What is claimed is:

1-20. (canceled)

21. An in vivo method of treating infections, comprising the steps of:
providing an encapsulated biocidal agent comprising an outer coating layer on a liposomal carrier having nanoparticles or nanostructures of biocidal material encapsulated within said liposomal carrier, and
introducing said encapsulated agent into the bloodstream of a mammalian host, said mammalian host having an infection at one or more inflammation sites, wherein said encapsulated biocidal agent travels in said bloodstream to reach said inflammation site.

22. The method of claim 21, wherein macrophages at said inflammation site engulf and dissolve said outer coating layer and said liposomal carrier to free said biocidal material.

23. The method of claim 21, wherein said biocidal material remains in said bloodstream for more than one day.

24. The method of claim 21, wherein said outer coating layer comprises polyethylene glycol (PEG).

25. The method of claim 21, wherein said encapsulated biocidal agent, is <150 nm in size.

26. The method of claim 21, wherein said biocidal material comprises elemental silver, elemental copper, or elemental mercury.

27. The method of claim 26, wherein said elemental silver is nanoparticle colloidal silver.

28. The method of claim 21 wherein said biocidal material comprises iodine.

29. The method of claim 21, wherein a size of said encapsulated biocidal agent is less than or equal to 85 nm in size.

30. The method of claim 29, wherein said size is less than or equal to 10 nm in size.

31. The method of claim 21, wherein said infection is induced by a pathogen.

32. The method of claim 31, wherein pathogen comprises an antibiotic-resistant bacteria, a biological warfare agent resistant to antibiotic therapy by natural adaption, human selection or human engineering, a nosocomial infectious agent, a virus or a fungus.

33. The method of claim 21, wherein said introducing step comprises directly injecting said encapsulated biocidal agent into said bloodstream.

34. The method of claim 21, wherein a concentration of said biocidal material within said encapsulated biocidal agent is at least 0.45 ppm without being toxic to said mammalian host.

35. The method of claim 21, wherein said mammalian host is a human host.
